(19) United States
(12) Patent Application Publication
(10) Pub. No.: US 2017/0326311 A1
(43) Pub. Date: Nov. 16, 2017

(54) Device for Fit Testing of Gas Masks

(71) Applicant: Nextteq LLC, Tampa, FL (US)

(72) Inventors: Bryan I. Truex, Tampa, FL (US); Gueorgui M. Mihaylov, Virginia Beach, VA (US); Ryan Day, Riverview, FL (US)

(73) Assignee: Nextteq LLC, Tampa, FL (US)

(21) Appl. No.: 15/593,503

(22) Filed: May 12, 2017

Related U.S. Application Data
(60) Provisional application No. 62/335,130, filed on May 12, 2016.

(51) Int. Cl. A61M 11/00; G01M 3/26; A62B 27/00

(52) U.S. Cl. CPC A61M 11/006; G01M 3/26; A61M 11/00; A62B 27/00

(57) ABSTRACT

Fit testing is conduct to ensure that gas mask provide proper protection to workers that may be exposed to airborne contamination. Standard fit test procedures require the mixing of fit test compounds to specific fit testing composition required for threshold testing and subsequent fit testing. A device is needed for easy mixing of the components of the fit test composition and easy delivery of fit test composition during a fit test. For example, a device for fit testing of a gas mask on an individual wearer may include a sprayer, at least one ampoule containing a fit testing component, and a reservoir containing the ampoule and a solvent for the fit test component. The reservoir may be hollow body having a sealed inner volume. The reservoir may be deformable to break at least one hermetically sealed ampoule without tearing or otherwise breaking the reservoir. Therefore, the ampoule may be broken within the reservoir to mix the gas mask fit testing component into the solvent to generate the desired concentration for either a threshold test or gas mask fit test.

### FIELD OF INVENTION

Industrial workers wear respiratory protective devices (hereinafter “gas masks”) to avoid and reduce inhalation of airborne contaminants in many industrial situations. An estimated five million workers wear some type of respirator, either occasionally or routinely. Though it is generally preferred to use engineering controls to reduce airborne contaminant emissions at their source, there are operations and environments where personal protective equipment may be required.

Gas masks provide adequate protection only if the gas mask is properly selected for the airborne contaminants, the proper filter/canister is attached, fits the wearer properly, and is properly put on and worn. Gas mask fit testing can confirm that the gas mask fits the face of the wearer. Embodiments of the device for fit testing of gas masks may be used in methods and procedures to test the fit of a gas mask on a particular wearer. Embodiments of the methods of gas mask fit testing describe herein conform to the requirements of existing industry standards for fit testing of gas masks. Embodiments of the device may create a fine mist of a solution comprising at least one component that indicates whether the gas mask fits and seals properly over a wearer's face by eliciting a response or other indication from the wearer that the gas mask fit test component bypassed the gas mask canister due to a poor fitting gas mask.

### BACKGROUND OF THE INVENTION

Gas masks may not provide effective protection for all wearers. For example, individuals with impaired lung function may be physically unable to breathe properly or draw sufficient air through a gas mask canister or other filter or facial hair on a wearer may interfere with the seal between the gas mask and the face of the wearer allowing potentially contaminated air to by pass the canister or other filter. Without an effective seal between the gas mask seal and the face of the wearer, an individual will be unable to obtain effective protection from a gas mask that relies upon a tight-fit to provide protection.

Problems with the fit of a gas mask may result in reduced protection from contaminants. Occupational safety and health personnel have spent considerable time and effort developing fit-testing procedures and methods of measuring respirator effectiveness for individual wearers.

In facilities where respirators are required to be worn, regulations require that a written respirator protection program must be developed and implemented in accordance with OSHA's respirator standard, 29 CFR 1910.134. The respirator program must include step-by-step instructions for each procedure so that a task (i.e., respirator use, fit-testing procedures, cleaning and storage, etc.) can be performed by personnel in a uniform and consistent way, while providing the maximum protection for workers who may have to use a respirators in the workplace.

The respiratory protection program should include gas mask fit testing procedures. Several methods of fit testing have been developed to assure acceptable fit test of the gas masks over the human face. Some of these methods have been approved by OSHA as standards for gas mask fit testing. One step common to these accepted fit testing methods is creating an aerosol or mist of a chemical compound by evaporating a liquid or by generating an aerosol of micro-particles of a solution of the irritant or other indicating compound or compounds. With a canister attached to a properly fitted gas mask, the canister will remove the indicating compounds from the breathable air and they will not be detected by the wearer. However, if the gas mask is not properly fitted and there is a leak in the seal between the mask and the face, the indicating air may enter the inner volume of the mask and be sensed by the wearer as a taste (bitter or sweet, for example) or as an irritant. If the vapors or micro-particles are identified by wearer as a known smell, taste or an irritation occurs at relatively very low concentration outside of the mask, a leak must be present and the test indicates an inadequate fit to the face and an ineffective seal.

Recently several kits of fit testing equipment have become available that if used properly satisfy the requirements of the standards set by OSHA and NIOSH and indicate the effectiveness of the gas mask seal. For example, fit testing with aromatic banana oil is typically performed with a paper towel wetted with, the banana oil. The banana oil evaporating from the paper towel creates a detectable odor of banana oil in the area around the seal of the gas mask. Any breach of the banana oil into the inner volume of the gas mask may be easily sensed.

Two other OSHA compliant fit tests use saccharin and Bitrex. Saccharin and Bitrex are solid substances but may be dispersed in air through aerosols. To create a fit testing aerosols of these substances, a solution comprising either the saccharin and/or the Bitrex is “atomized” with a sprayer or a nebulizer. The air inside a nebulizer may be pressurized by a squeezable rubber bulb connected to the device similar to a perfume atomizer.

A fine mist of the solution comprising the saccharin or the Bitrex is created and the solvent is quickly evaporated resulting in a particulate aerosol, of the saccharin or the Bitrex.

The drawbacks of these fit testing kits, devices, and methods are several including:
The conventional sprayers require use of both hands of the fit test personnel to operate, one hand to hold the reservoir and the other hand to squeeze the atomizer bulb;
The sprayer and the rubber bulb are comparatively bulky objects that may be difficult to manipulate during a test;
The nozzle of the sprayers may easily clog by the dry components of the aerosol when the solvent evaporates as the solution leaving the dried components to accumulate at the nozzle of the conventional fit test sprayers. The dried substance, BITREX, saccharin, or banana oil, for example, accumulates at the sprayer nozzle and will reduce efficiency of the sprayer then eventually finally clogging the nozzle. The sprayer must be carefully cleaned immediately after each fit test to maintain consistent reliable fit testing and prevent clogging;
The conventional sprayers in gas fitting testing kits (which were designed for lung inhalation, not fit testing) entrap a portion of the mist in the delivery tube reducing the reliability and consistency of the fit testing. The conventional sprayers were actually designed for asthma relief and thus to direct the aerosol flow for delivery to the trachea and to avoid entrapping the substances onto the mouth and tongue surfaces and are, therefore, not ideal for fit testing purposes;
According to the standardized accepted methods for these fit testing procedures, all solutions have to be freshly prepared to specific threshold and fit test concentrations. Small vials are used to store preliminary prepared solutions of the components of the tests. The procedure involves unplugging the vial or breaking the breakable end of ampoule, preparing the complete fit test solution, and then transferring the liquid into the base of the sprayer, sealing the sprayer and spraying the fit or threshold testing solution in the fit test area. The series of small preparations steps makes the entire process tedious, prone to error, and time consuming. Since each testing session requires fresh solutions, there is a significant risk that the fit test solutions will be consistently prepared resulting in inconsistent fit testing and potentially not indicating ineffective seals.

There is a need of fit test device that avoids at least a portion of the drawbacks of conventional devices and processes. There is also a need for a gas mask fit testing kit that provides simple and convenient preparation and delivery of fit testing solutions.

### SUMMARY OF THE INVENTION

In some embodiments, a device for fit testing of a gas mask on an individual wearer comprises a sprayer, at least one ampoule containing a fit testing component, and a reservoir containing the ampoule and a solvent for the fit test component. The ampoule comprises the amount of fit testing component to dissolve within the solvent to properly produce either a threshold fit test solution or a gas mask fit testing solution.

The reservoir may be hollow body, in some embodiment the reservoir may be a cylindrical tubular shape, and/or made from a deformable material such as a deformable plastic or other polymeric material. The reservoir may be deformed to break at least one hermetically sealed ampoule without tearing or otherwise breaking the reservoir. In one embodiment, the reservoir may be squeezed such as between a thumb and index finger, for example, to deform the reservoir and break the ampooule within the reservoir without opening the reservoir. The reservoir may also comprise an appropriate solvent for the fit test component in the ampoule. Therefore, the ampoule or ampoules may be broken within the reservoir to mix the gas mask fit testing component into the solvent to generate the desired concentration of a testing solution for either a threshold test or gas mask fit test.

Further, the device may comprise a sprayer, nedulizer, or other aerosol creating device for development and delivery of a mist of the solvent and gas mask fit test component. The sprayer or other aerosol creating device may include a pump (manual or electric), an aerosol created from pressurized gas or air from a cylinder or aerosol spray can (similar to air freshener or a CO2 cartridge, such as used to inflate life vests). Naturally, the pressurized air-based system will need some type of trigger mechanism to release the pressure.

Further, the reservoir may contain one or more additional breakable ampoules. For example, one of the ampoules may contain a substance for generating carbon dioxide gas (for example, sodium bicarbonate within the ampoule may react with an acid within the reservoir to generate the carbon dioxide) in contact with a component of the solvent. In one embodiment, the solvent may comprise water, a water sodium chloride solution, or a water and ethanol solution, for example. The solvent may further comprise an acid, for example, a weak citric acid that will react with the carbonate or bicarbonate to generate carbon dioxide. The generated carbon dioxide in the reservoir will increase the pressure in the reservoir over the atmospheric pressure.

In another embodiment, the reservoir may contain an ampoule comprising the fit test component and a second ampoule containing the solvent. Both ampoules must be opened to produce the fit test solution or the threshold test solution. In such an embodiment, there may be additional ampoules containing the gas generating components or gas generating components may be contained in either the fit test component ampoule, the solvent ampoule, or both based upon the compatibility and stability of the components.

In some embodiments, the exit nozzle of the sprayer may be rotatable 360* and may be pointed to any desired direction. A gasket, O-ring, adhesive or other sealing means may be situated into the assembly between the reservoir and a cap for sealing the reservoir with an effective air tight seal for storage of the device. The reservoir and the cap, in some cases the cap comprises a pump sprayer, may be releasably connected by a snap fit, interference fit, or threaded connection, for example. For an unpressurized reservoir, in storing mode the thread connection is air tight to increase shelf life. However, during testing, the cap may be loosened to allow air into hollow body, thereby, to compensate for the volume of liquid sprayed out and the resultant reduced pressure.

In the embodiments, the device comprising two or more seated ampoules with components that could cause generation of pressurizing gas such as, but not limited to, carbon dioxide, allows spraying with greater force, leading to a fine mist when sprayed. The ampoules may be contained with the reservoir in one line or in flat or oval cross-section body close to each other. In any position, the reservoir is configured to allow the user to break the ampoule or ampoules by bending or otherwise manipulating the device, as described above, and allow the fluids contained in the ampoule or ampoules to mix with the solvent allowing in situ preparation of a fresh batch of gas mask test solution with the desired concentration for either a threshold testing or a gas mask fit testing.

In another embodiment, a device for fit testing of a gas mask on an individual wearer comprises a sprayer, at least one ampoule containing a fit testing component in a solvent, and a reservoir containing the ampoule. In this embodiment, the reservoir does not contain a separate solvent, however, the ampoule comprises a fit testing composition comprising both the fit test component and the solvent. The reservoir may be deformed to break the at least one hermetically sealed ampoule without tearing or otherwise breaking the reservoir. As in the other embodiments, the ampoule may be sold within the reservoir or the ampoule sold separately and added to the reservoir as needed.

In another embodiment wherein all the fit test composition components are within ampoules, one ampoule may comprise the fit test component and the second ampoule may comprise a solvent. Therefore, the ampoules may be each broken within the reservoir to release the components and mix the gas mask fit testing component into the solvent to generate the desired concentration for either a threshold test or gas mask fit test.

The threshold concentration of the fit test component may be in any effective concentration that is capable to confirm that the test subject can sense the presence of the fit test component at low concentrations. For example, the concentration of the fit test component in the threshold test solution may be between greater than 0 wt. % and less than 3 wt. %.

The fit test concentration of the fit test component may be in any effective concentration that is capable to confirm that the test subject can sense a leak in the gas mask seal during the fit test by sensing the presence of the fit test component within the inner volume of the gas mask. The concentration of the fit test component in the fit test solution is generally higher than the concentration of the fit test component in the threshold test. The concentrations in both solutions depends on the concentrations of the fit test component that will elicit a response from the test subject, first, to indicate a low level response to the fit test component (threshold) and second, to indicate a small leak in the gas mask seal with the wearer's face. The fit test concentration, therefore, may be in a higher concentration than the threshold test, for example, the fit test component may be in the range of 30 wt. % to 100 wt. %.

The concentrations for fit testing and threshold testing are indicated in OSHA regulations and procedures. The fit test devices may include concentrations in a range from above or below these concentrations by 33% in some embodiments, for example. In other embodiments, the fit test devices may comprise concentrations in a range above or below the fit test concentrations and the threshold concentrations by 25% and, in still further embodiments, the fit test devices may comprise concentrations in a range above or below the fit test concentrations and the threshold concentrations by 10%.

In further embodiments, the design of the device may be used with one hand and is more convenient than the convenient sprayers requiring two hands.

Additionally, embodiments of the device reduce the time necessary to prepare the fit test and threshold test concentrations prior to testing while having a compact, disposable and low cost design.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of components, parts, techniques and steps are disclosed. Each of these has individual benefit and each can also be used in conjunction with one or more, or in some cases, all of the other disclosed embodiments and techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

### BRIEF DESCRIPTION OF THE DRAWINGS

FIG. 1 depicts a gas mask testing device comprising a reservoir 12, the reservoir 12 contains a single ampoule 22 filled with a gas mask testing component (for example, saccharin, isoamyl acetate, and/or denatonium benzoate) and a solvent, the solvent may be a water/alcohol solvent; the alcohol may be ethanol, for example;

FIG. 2 depicts the device with a first ampoule 42 and a second ampoule 43; the first ampoule may contain saccharin, isoamyl acetate, non-toxic, irritating, weak acid solutions with a pH of 3 or greater such as hydrochloric acid or phosphoric acid, or denatonium benzoate and the second ampoule may contain a weak solution of sodium bicarbonate, for example. The solvent in the reservoir may contain a weak water solution of citric acid in water or merely water in the embodiment wherein the first ampoule contains an acid; therefore, when both ampoules are broken open, the bicarbonate will react with the citric acid solution or other acid to release CO2 and neutralize the acidity of the solvent to neutral solvent, resulting in an increase in pressure in the reservoir above the normal atmosphere pressure; and

FIG. 3 depicts an embodiment of the device wherein the reservoir comprises a bendable section or an accordion section 50 that allows the reservoir to be bent to break the ampoule within the reservoir.

### DESCRIPTION

Gas mask fit testing is required to ensure adequate protection of personnel in areas that may potentially be contaminated with toxic substances. The protection provided by a gas mask may be limited by improper fit of the gas mask to a specific user's face. The Occupational Safety and Health Association (“OSHA”) has defined fit testing procedures and many companies sell gas mask fit testing kits comprising the components necessary to perform these testing procedures.

The OSHA approved procedures include procedures for using isoamyl acetate, saccharin solution, stannic chloride, and denatonium benzoate solution in aerosol as gas mask fit test components to determine the fit of a gas mask. These procedures describe specific threshold concentrations of these gas mask fit test components for preparation of a threshold testing solution and a second testing concentration of these gas mask test components for the actual gas mask fit testing. The threshold concentrations are typically a lower concentration and used to determine whether the subject has an identifiable reaction to the gas mask fit test component. The reaction is typically an irritation, distinct taste or distinct odor, for example.

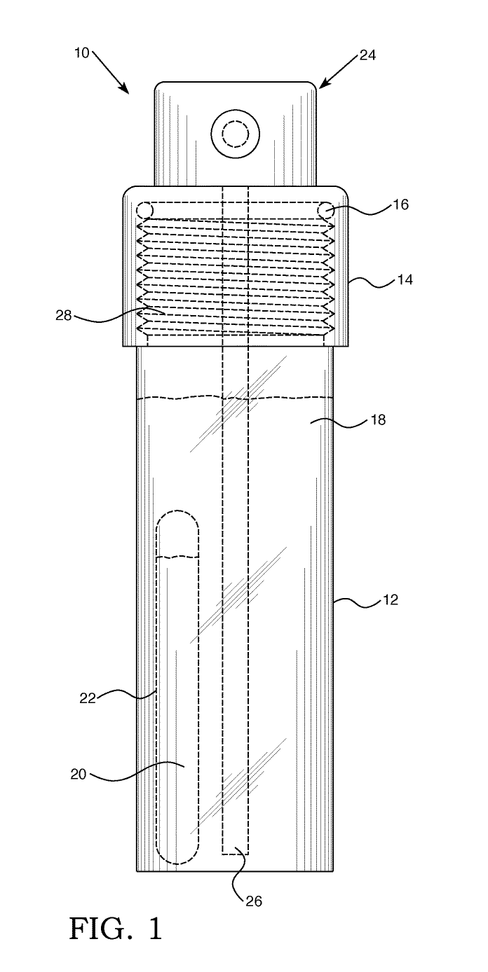

Embodiments of a gas mask fit testing sprayer may be used to prepare accurate and consistent threshold and fit testing concentrations of gas mask test components for OSHA approved fit testing procedures. Embodiments of the gas mask fit testing sprayer comprise a reservoir for containing a fit testing composition and a cap creating an effective air tight seal on the reservoir. The reservoir contains a solvent for the gas mask fit test component. The reservoir and the cap create an air tight seal for maintaining the quality of the solvent within the reservoir and improve the shelf life of the testing components. From the storing sealed mode, the cap may be twisted to equalize the pressure in and out of the reservoir (¼ turn may be sufficient, for example, for some embodiments.) The sprayer 10 may be shaken to mix and allow equilibrium of the concentration of gas mask test component into the solvent. One stroke normally expels 100 microliters of liquid and 10 strokes may be needed for one test level and this may be extended to 30 strokes that may be needed to complete the test. In some embodiments, the fit testing device 10 comprises the reservoir 12 and the ampoule 22 have enough volume for several consecutive tests as the requirements are for freshly prepared solutions. See FIG. 1.

As used herein, an “air tight seal” is a seal that sufficiently prevents air infiltration and degradation of the contents of the reservoir of the fit testing device resulting in a shelf life of over three months.

As used herein, a fluid may be a liquid or a gas and includes solids suspended in a liquid or a gas.

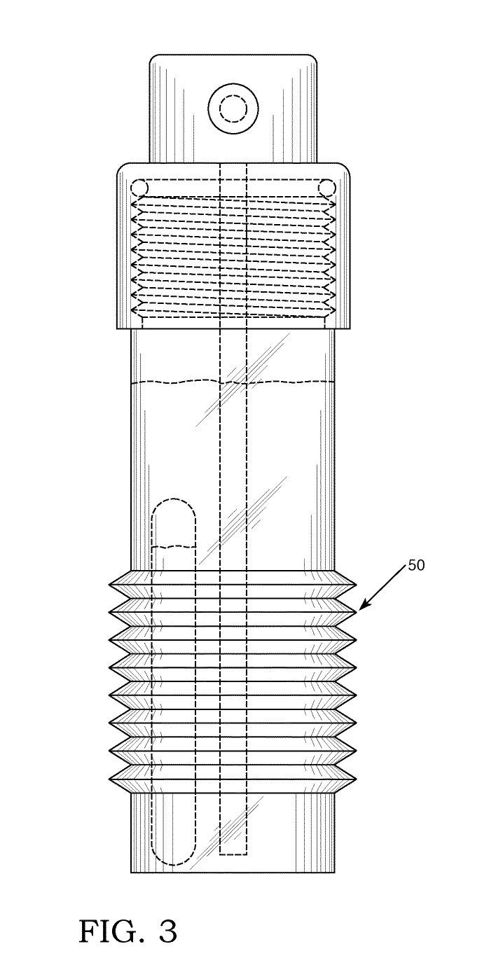

The reservoir also contains an ampoule containing a liquid. In one embodiment, a first sealed ampoule contains a gas mask fit test component or a precursor component that reacts with a component in the solvent to form a gas mask fit test component. Prior to use, the walls of the ampoule separate the gas mask fit test component from the solvent and the gas mask fit test component is soluble in the solvent, wherein a breakable wall of the ampoule separates the solvent from the gas mask fit test component or the precursor component. The sealed ampoule may comprise breakable walls. In such an embodiment, the reservoir may comprise resilient walls such that the sealed ampoule may be crushed or cracked while within the reservoir. As shown in FIG. 3, the resilient walls may further comprise a bendable section 50 such as, but not limited to, an accordion section, a hinge, or an articulated portion, for example. In a specific embodiment, the reservoir may comprise two bendable sections 50 such that the neck of a first ampoule may be snapped off with the first bendable section and the neck of a second ampoule may be snapped off with the second bendable section. In such embodiments, the first ampoule may comprise the specific quantity of fit test component for a threshold test and the second ampoule may contain the additional quantity of fit test component to prepare the fit test composition as described by OSHA, other regulatory agency or other protocol.

The gas mask fit test component, the solvent and any other additional components may be mixed after being released from the ampoule into the reservoir. The gas mask test kit may further comprise a sprayer capable of creating a mist of the solution comprising the solvent and the gas mask fit testing component to provide the test environment with a sufficient amount to gas mask test solution for threshold testing and gas mask fit testing.

Reservoir and Ampoule

The reservoir may be any container capable of being sealed to receive and contain at least one ampoule. The reservoir may further contain a solvent. The ampoule may separate one set of components within the reservoir from the other or may merely provide a longer shelf life for the fit test component or a fit test composition within the ampoule. A set of components may include at least one of the compound selected from the group comprising a gas mask fit test component, gas mask fit test component precursors, a solvent, ethanol, water, gas inert to the other components, gas producing reactants such as carbonates, bicarbonates, weak acids, citric acid, acetic acid, hydrochloric acid, phosphoric acid, and other components. In an embodiment, a solvent is contained within the reservoir of a gas mask fit test sprayer and a gas mask fit test component is contained within a breakable ampoule. Breaking the ampoule results in mixing of the solvent and the gas mask fit test component produces the gas mask test solution. In another embodiment, the solvent is contained in a breakable ampoule within the reservoir of a gas mask fit test sprayer or device and a gas mask fit test component is contained within a breakable ampoule with the reservoir of the gas mask fit test sprayer or device.

The reservoir may be sealed with a cap creating an effective air tight seal on the reservoir. The cap may be connected to the reservoir with a threaded connection, an interference fit, a snap connection and/or a bayonet connection, the connection may include an adhesive to create the air tight seal, for example. The air tight seal may include a gasket, an O-ring, sealing grease, adhesive, or caulking, for example. In some embodiments, the effective air tight seal is created by an adhesive connecting the cap to the reservoir and, in such embodiments, it may be preferred to have the reservoir pressurized or to have pressurizing reactants in at least one ampoule and/or in the reservoir.

In other embodiments, the reservoir may not be sealed. For example, the reservoir may be a pipette with an ampoule in the bulb or a container with a spout or other opening.

The ampoule may be constructed of any material that may be broken within the reservoir to release its contents. The ampoule may be made from glass and/or plastic, for example. The glass or plastic ampoule may be colored to retard certain wavelengths of light penetration into the ampoule. Such embodiments may be useful for compounds within the ampoule that may be sensitive to exposure to light. Further, the reservoir may also be a colored or opaque material to complement the light protection of the glass or plastic ampoule.

The ampoules may also be color coded to indicate the fit test component or solvent within the ampoule. The ampoules may further comprise indicia indicating the ampoule required to be broken for the threshold test or the fit test. The ampoules may include numbering indicating the order of breaking the ampoules to within the reservoir to first perform the threshold test and then to break the second ampoule to perform the fit test. In another embodiment, the ampoule containing the amount of fit test component for the threshold test may be longer than the ampoule containing the additional amount of fit test component required for the actual gas mask fit testing. In this embodiment, the ampoule for threshold testing is easier to break first and more apparent to the user.

The ampoules and/or the reservoir may include identifying information coded in a bar code, QR Code, data matrix, or other computer readable indicia print on their surface. The identifying information may include, but are not limited to, a part number, manufacturer, date of manufacture, shelf life expiration date, fit test component, quantity of fit test component, ratio of other components necessary to prepare a fit test composition, threshold test component, quantity of threshold test component, threshold test solvent, quantity of threshold test solvent, environmental operating specifications, and environmental storage conditions, for example.

Gas Mask Fit Test Components

Gas mask fit test components include saccharin, denatonium benzoate (BITREX™), stannic chloride, isoamyl acetate, non-toxic, irritating, weak acid solutions with a pH of 3 or greater such as hydrochloric acid or phosphoric acid, combinations thereof or other compounds that a test subject may clearly detect the presence of through odor, taste, or mucus membrane irritation, for example.

The reservoir or the ampoule may contain gas mask fit test component precursors. For example, isoamyl acetate may be prepared from the reaction of between isoamyl alcohol and glacial acetic acid. The acetic acid may be in the solvent in contained in the reservoir and the isoamyl alcohol contained in the ampoule or other combinations of ampoules and components in the reservoir.

In one embodiment, the gas mask fit test sprayer comprises a first sealed ampoule containing a gas mask fit test component, wherein the sealed ampoule is within the reservoir. The ampoule comprises breakable walls and the breakable walls of the ampoule separate the gas mask fit test component from the solvent. In such embodiments, the gas mask fit test component is soluble in the solvent. The gas mask fit test component may be a solid or a liquid. The gas mask fit test component is present in the desired ratio to the solvent within the reservoir to prepare the required concentrations for a threshold test or gas mask fit test as defined in OSHA regulations. For example, for a fit test with denatonium benzoate, a threshold check solution is prepared by adding 13.5 milligrams of denatonium benzoate to 100 ml of 5% salt (sodium chloride, NaCl) solution in distilled water. This ratio may be used to prepare other volumes of fit testing solutions.

An embodiment of a gas mask fit test sprayer is shown in FIG. 1. The gas mask fit test sprayer of FIG. 1 comprises a reservoir 12 having resilient walls. The reservoir 12 is sealed with a cap 14 by a threaded connection 28. In the depicted embodiment, the cap also comprises a sprayer and pump 24 and a dip tube 26 to draw fluid from the reservoir 12 into the sprayer and pump 24. A typical sprayer would not have an air tight seal between the cap 14 and the reservoir 12 but would allow air to enter the reservoir through the connection between the reservoir and the cap. This allows air to enter the reservoir to replace liquid that is pumped out by use of the sprayer. However, the gas mask fit test sprayer of FIG, 1 comprises an O-ring 16 between the reservoir 12 and the cap 14 to improve the shelf-life of the gas mask fit test components and to prevent the components from escaping prior to or after the threshold or fit testing procedures. If the sprayer no longer functions due to reduced pressure, the cap may be loosened to allow air to enter the reservoir and the pump to again function properly. In other embodiments, the reservoir may be pressurized to assist in spraying. The reservoir may be pressurized by, for example, adding a pressurized inert gas to the reservoir during manufacture or prior to use, initiating a gas producing chemical reaction between chemicals in the ampoule 22 and in the reservoir, or having a pressurized gas within the ampoule 22 that is released when the ampoule is broken.

The gas mask fit test sprayer comprises an ampoule 22 within the reservoir 12. In the embodiment shown, for a threshold test, the ampoule 22 comprises a fit test component 20 of 13.5 milligrams of denatonium benzoate per 100 ml of 5% sodium chloride solution in water in the reservoir 22 to threshold testing or, for a gas mask fit test, the ampoule contains 337.5 mg of denatonium benzoate per 200 ml of a 5% sodium chloride solution in water in the reservoir 22. The sodium chloride is a solvent 18 for the gas mask fit testing component 20, denatonium benzoate in the ampoule 22.

For a threshold test using banana oil, a solution is prepared using 1 ml of isoamyl acetate per 800 ml of odor-free water.

For a gas mask fit test using saccharin, a threshold check solution may be prepared by dissolving 0.83 gram of sodium saccharin USP per 100 ml of warm water. Alternatively, the threshold check solution may be prepared by using 1 ml of the saccharin fit test solution per 100 ml of distilled water. A saccharin fit test solution may be prepared by using 83 grams of sodium saccharin per 100 ml of warm water. To prepare the saccharin fit test solution with the gas mask fit test sprayer of FIG. 1, the 10 ml sprayer reservoir may be filled with 8 ml of water and the ampoule contains 6.64 grams of sodium saccharin. When the ampoule is opened, the sodium saccharin and the water may be mixed to form the proper fit test solution. The fit test solution may be used for a number of fit tests and the sprayer may be disposed by the appropriate methods.

In some embodiments, the reservoir may contain a threshold test solution comprising an appropriate concentration of fit test component and an ampoule within the reservoir may contain the additional fit test component to convert the threshold test solution to a gas mask fit test solution.

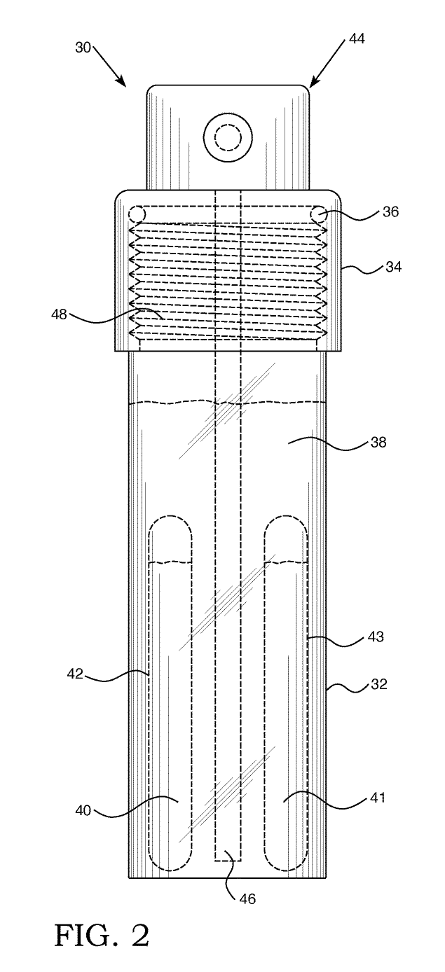

Another embodiment of gas mask fit test sprayer is shown in FIG. 2. The embodiment of the gas mask fit test sprayer of FIG. 2 comprises a reservoir 32 having resilient walls. The reservoir 32 is sealed with a cap 34 by a threaded connection 48. The cap 34, similar to the embodiment shown in FIG. 1, also comprises a sprayer and pump 44 and a dip tube 46 to draw fluid from the reservoir 32. The gas mask fit test sprayer of FIG. 2 comprises an O-ring 36 between the reservoir 32 and the cap 34 to improve the shelf-life of the gas mask fit test components. The O-ring may be replaces with any of the sealing devices or means described above.

The gas mask fit test sprayer comprises a first sealed ampoule 42 and a second sealed ampoule 43 within the reservoir 32. In one of the possible embodiments for a gas mask sprayer comprising two sealed ampoules, the first sealed ampoule may contain the proper amount of gas mask fit test component to prepare a threshold test solution. In such embodiment, the second sealed ampoule 43 contains the additional gas mask fit test component to prepare the fit test solution in addition to the gas mask fit test component of the first sealed ampoule. Therefore, the first sealed ampoule may be opened to prepare the threshold test solution and both the first sealed ampoule and the second sealed ampoule may be opened to produce the fit test solution.

Alternatively, a fit testing device 30 may comprise the first sealed ampoule 42 containing the gas mask fit test component 40 and the second sealed ampoule 43 may contain a pressurizing component or a component 41 that reacts with a component in the solvent 38 or in the first sealed ampoule 42 to produce a pressurizing gas within the reservoir 32. For example, a second sealed ampoule, wherein the second ampoule contains a gas generating compound, wherein the gas generating compound reacts with a reactive gas generating component of the solvent 33 or a component of the first sealed ampoule. In some embodiments, for example, the gas generating compound may be at least one of a carbonate and a bicarbonate and the reactive component is an acid. The carbonate or bicarbonate may react, with the acid to form carbon dioxide gas. The sealed reservoir 32 becomes pressurized by generation of the gas. The acids include, but are not limited to, at least one of citric acid, hydrochloric acid, phosphoric acid, and acetic acid. If compatible, the fit test component may be mixed with a gas generating component in one or more than one ampoule. The reactive components may be contained directly in the solvent 33 in the reservoir 32 or In a separate ampoule 42 43.

More generally, a reaction between a gas generating compound in at least one of the first ampoule, the second ampoule, or the solvent and a reactive component in another one of the first ampoule, the second ampoule, or the solvent form a gaseous component within the reservoir thereby increasing the pressure within the reservoir. The seal, such as O-ring 36 and threaded connection 48 on cap 34, for example, between the reservoir 32 and the cap 44 results in pressurization of the reservoir and is sufficient to retain the increased pressure. The reservoir 32 comprises means for breaking the breakable walls of the ampoule. The means for breaking the ampoule may include resilient walls or a mechanical device. In one embodiment, the reservoir comprises resilient walls that may be squeezed or the reservoir may be bent to break the breakable walls or breakable joint of the ampoule. The reservoir may further comprise a mechanical device such as, but not limited to, a plunger, threaded connection to reduce the size of the reservoir to break the ampoule, or other mechanical means to break the ampoule.

In a still further embodiment for the device for fit testing of a gas mask on an individual wearer, all the fit test composition components are within ampoules. The first sealed ampoule may comprise the fit test component. The fit test component may be the saccharin, denatonium benzoate, isoamyl acetate, and non-toxic, irritating, weak acid solution with a pH of 3 or greater including but not limited to hydrochloric acid and phosphoric acid, for example. As in the other embodiments, the fit test component may be a solid, liquid, or a solid dissolved in a solvent. In such embodiments, the second ampoule may comprise a solvent, a pressurized gas such as, but not limited to, air, nitrogen, an inert gas and/or carbon dioxide, and/or gas creating reactants. Though the reservoir does not directly contain the solvent or the fit test component (these components are contained within the ampoules which are contained within the reservoir), the reservoir may directly contain a gas generating component such as the carbonate or bicarbonate, for example. Therefore, the ampoules may be each broken within the reservoir to release the components and mix the gas mask fit testing component into the solvent to generate the desired concentration for either a threshold test or gas mask fit test.

In some embodiments, for example, in embodiments wherein the fit test component or other component of the fit test kit is light sensitive, the ampoule may comprise a colored wall, reflective surface, or other light or ultraviolet light blocking material, coating or surface.

OSHA-Accepted Fit Test Protocols describe concentrations of fit test components for both threshold odor screening and fit testing. In some embodiments of the fit test device, the fit test device comprises a reservoir, wherein the reservoir contains an ampoule. The ampoule contains a premixed fit test composition. The fit test composition may comprise a concentration of a threshold test concentration or a fit test concentration of a fit test component. The fit test component may be any fit test component including, but not limited to, saccharin, denatonium benzoate, isoamyl acetate, and non-toxic, irritating, weak acid solution with a pH of 3 or greater including but not limited to hydrochloric acid and phosphoric acid, for example. The concentrations of the premixed threshold test concentration or fit test concentration may be the concentrations specified in the OSHA fit test procedures or other fit test protocol as described herein and in OSHA regulation §1910.134 and the associated Appendices.

Sprayers

The gas mask test kit may further comprise a sprayer capable of creating a mist of the solution comprising the solvent and the gas mask fit testing component to provide the test environment with a sufficient amount to gas mask test component for threshold testing and gas mask fit testing. Embodiments of sprayers may typically comprise a pump, a pressurized gas such as, but not limited to, air, nitrogen or carbon dioxide. Therefore, in some embodiments the gas mask fit test sprayer may comprise a pump sprayer. The pump sprayer, in some embodiments, may be attached to the tube that draws fluid from the reservoir into the pump. In addition, the pump forces the liquid out of the pump spray exit nozzle. Typically, a spray bottle comprises a pump designed to pull liquid from the reservoir through a one-way valve. Subsequently, the pump may be used to force the liquid through a nozzle that breaks up the flow of the liquid, turning it into a fine mist or stream. The sprayer is capable of creating a mist of the solvent and the gas mask fit testing component. In some embodiments, the solvent quickly evaporates from the mist leaving, an aerosol of the gas mask fit testing components.

The sprayer may also comprise a pressurized gas. The pressurized gas may be within the reservoir to provide a driving force for the fit test composition, may be in a cartridge attached to the reservoir for pressurizing the reservoir, or may be used to drive an aspirator nozzle with the Venturi effect.

The reservoir may have a screw-on cap, a snap on cap, or a bayonet fitting cap. The cap may be made of aluminum, metal, or plastic, for example. Some embodiments, the spray bottle may have a sprayer in the cap. The sprayer in the cap may have two one-way valves, one valve is between the pump and the reservoir and another one-way valve is between the pump and the nozzle. The one-way valve system causes the liquid to move in only one direction, from within the reservoir and out the exit nozzle. The one-way valves typically comprise a small piece of rubber to regulate the flow, such as a small ball. One ball is placed in the valve between the pump and the reservoir and the other one in the valve between the pump and the nozzle.

The reservoir may not comprise a sprayer. For example, in embodiments of the device for fit testing with isoamyl acetate, the OSHA test comprises applying the fit test component to a paper towel, such as, for example, a 6-inch by 5-inch piece of paper towel, or other porous, absorbent, single-ply material, folded in half and wetted with 0.75 ml of pure IAA. The reservoir may just comprise a cap, a releasable top, another opening, or the reservoir may be already open such as a pipette. The ampoule may be broken within the reservoir, such as the pipette, and then applied to the paper towel.

Embodiments include a method of fit testing a gas mask on a wearer. One of the methods may comprise breaking a first sealed ampoule containing a gas mask fit test component within a reservoir to release the gas mask fit test component into the reservoir. The ampoule may contain sufficient gas mask fit test components for a threshold test or a fit test. The reservoir may comprise an air tight seal with a cap or the reservoir may be able to “breathe” as liquid is removed from the reservoir. Once released and mixed with solvent in the reservoir, the gas mask fit test component may be sprayed from the sealed or unsealed reservoir in an area around the person wearing a gas mask or under a hood used for fit testing. After spraying the gas mask component, the tester monitors the gas mask wearer to perceive an indication from the person that the person senses the gas mask fit test component within the gas mask.

Another embodiment of the method for testing the fit of a gas mask on a wearer may comprise breaking a second sealed ampoule within the reservoir to release a gas generating component into the reservoir, wherein the reservoir contains a solvent, the first sealed ampoule and the second sealed ampoule. In one embodiment, the first ampoule contains a reactive compound that reacts with the gas generating component to from a gas such as carbon dioxide. For example, the solvent may comprise a reactive compound that reacts with the gas generating component to from a gas.

In other embodiments, the fit test sprayer may comprise a reservoir and a separate sprayer or atomizer. An embodiment of a fit test kit may comprise a plurality of reservoirs, wherein the reservoirs contain sealed ampoules and the ampoules contain a fit test component. The sprayer may be selectively attached to each ampoule to allow atomization or creation of an aerosol of the fit test composition within the reservoir. The sprayer may be used with multiple reservoirs. The sprayer may comprise a tube that may puncture the reservoir as the sprayer or atomizer is attached to the reservoir. In some embodiments, the tube may also contact and break the ampoule as the sprayer is attached to the reservoir. Such a connection may be a bayonet connection, for example.

The gas mask fit test device or sprayer may include a one-way valve to allow air to enter the reservoir as the fit test composition is removed from reservoir as the fit or threshold tests are conducted. The one-way valve would allow air or other gas into the reservoir without letting the contents of the reservoir out through the valve. The valve may be incorporated into the cap, the sprayer, or the walls of the reservoir.

Banana Oil Fit Testing

A banana oil (isoamyl acetate) fit test comprises two steps. The first step is an odor test to indicate whether the test subject can discern between pure water and water comprising a low concentration of isoamyl acetate. According to OSHA preparation procedures, the odor test solution comprises 0.0001 vol. % of isoamyl acetate in water, a volume ratio of 1 part isoamyl acetate per 1,000,000 parts water. This is a low concentration and OSHA describes making a stock solution (0.125% isoamyl acetate in water) and using the stock solution to prepare the odor test solution. This odor test solution may be prepared in one step using the devices and methods described.

In one embodiment, a device for fit testing comprises a reservoir containing water. The reservoir may be any size. For example, the reservoir may contain between 0.5 ounces and 2 liters of liquid. In some smaller handheld embodiments of the fit test device, the reservoir may contain between 0.5 ounces and 16 ounces of liquid, or in still further embodiments, the reservoir may contain between 1 ounce and 8 ounces of liquid. In an example of an embodiment of a gas fit test device with a reservoir capable of containing 4 ounces (118 milliliters) of water and an ampoule. The reservoir may contain 100 milliliters of water and the ampoule may contain 0.0001 ml of isoamyl acetate (a volume ratio of 1 part isoamyl acetate per 1,000,000 parts water). The ampoule may be broken, within the reservoir to prepare the odor test concentration of isoamyl acetate. The device may or may not comprise a sprayer.

Since the fit test for isoamyl acetate uses pure isoamyl acetate, embodiments of the fit test device may comprise a reservoir that is empty but contains an ampoule containing pure isoamyl acetate. The ampoule may be broken to release the pure isoamyl acetate into the reservoir without dilution.

Bitrex

The fit test procedures for denatonium benzoate include a threshold test and a subsequent fit test. The threshold test is used to determine whether the test subject can recognize the bitter taste of the denatonium benzoate. According to the OSHA procedures, the threshold check solution is prepared by adding 13.5 milligrams of denatonium benzoate to 100 milliliters of a 5% sodium chloride solution in distilled water (0.135 milligrams denatonium benzoate per milliliter of 5% sodium chloride solution, a ratio of 1.35 milligrams of denatonium benzoate per liter of 5% sodium chloride solution). The fit test solution is prepared by adding 337.5 milligrams of denatonium benzoate to 200 milliliters of 5% sodium chloride solution in distilled water (1.6875 milligrams per milliliter of solution, a ratio of 1.6875 grams of denatonium benzoate per liter of 5% sodium chloride solution).

Embodiments of the fit test device may be prepared with various configurations. In one embodiment, the fit test and threshold test solutions are prepared in separate devices wherein the reservoir contains the 5% sodium chloride solution and an ampoule comprising the denatonium benzoate. For example, an embodiment of the fit test device may comprise a reservoir contain 50 milliliters of sodium chloride solution and an ampoule containing 84.375 milligrams of denatonium benzoate. Similarly, an embodiment of the threshold test device may comprise a reservoir contain 50 milliliters of sodium chloride solution and an ampoule containing 6.75 milligrams of denatonium benzoate. Therefore, no measuring is required by the fit test personnel and risk of error is reduced. The fit test personnel may merely break the ampoule within the fit test device's reservoir, shake the reservoir to mix the fit test component with the solution and begin testing.

In another embodiment, the fit test device may comprise a reservoir containing the 5% sodium chloride solution and two ampoules. An embodiment of the fit test device comprising a reservoir containing two amoules is shown in FIG. 3. A first ampoule may contain 6.75 milligrams of denatonium benzoate and when broken within the reservoir produces the threshold test concentration to confirm the test subject's reaction to the denatonium benzoate. Then the second ampoule containing 77.625 milligrams of denatonium benzoate may be broken to increase the concentration of the denatonium benzoate from the threshold concentration to the fit test concentration. The fit test may then be performed on the test subject.

Embodiments of the fit test device having a reservoir containing a fit test composition comprising denatonium benzoate may also have a sprayer or atomizer to produce an aerosol of the denatonium benzoate. An aerosol comprising a mist of solvent and denatonium benzoate is generated from the sprayer or atomizer. The solvent will evaporate resulting in airborne denatonium benzoate for the fit test or threshold test.

Saccharin

The fit test procedures for saccharin also include a threshold test and a subsequent fit test. The threshold test is used to determine whether the test subject can recognize the sweet taste of the saccharin when exposed to an aerosol containing the saccharin. According to the OSHA procedures, the threshold check solution is prepared by adding 0.83 grams of saccharin to 100 milliliters of warm water (8.3 milligrams saccharin per milliliter of water, a ratio of 8.3 grams of saccharin to liter of water). The fit test solution is prepared by adding 83 grams of saccharin to 100 milliliters of warm water (0.83 grams per milliliter of solution, a ratio of 830 grams of saccharin per liter of water).

Embodiments of the fit test device may be prepared with various configurations with one ampoule or two (or more if gas generating compounds are included, for example). In one embodiment, the fit test and threshold test solutions are prepared in separate devices wherein the reservoir contains the water and an ampoule contains the required amount of saccharin for either the fit test or the threshold test. For example, an embodiment of the fit test device comprising a reservoir containing 100 milliliters of water and an ampoule containing 83 grams of sodium saccharin. Similarly, an embodiment of the threshold test device, the reservoir may contain 100 milliliters of water and an ampoule within the reservoir may contain 0.83 grams of sodium saccharin. Again, no measuring is required by the fit test personnel to prepare the fit test solution or the threshold test solution and risk of error is reduced. The fit test personnel may merely break the ampoule within the fit test device's reservoir, shake the reservoir to mix the fit test component with the solution and begin testing.

In another embodiment, the fit test device may comprise a reservoir containing water and two ampoules. A first ampoule may contain 0.83 grams of saccharin and when broken within a reservoir containing 100 ml of water produces the threshold test concentration for saccharin to confirm the test subject's reaction to the sweetened aerosol. Then the second ampoule containing 82.17 grams of saccharin may be broken to increase the concentration of the saccharin from the threshold concentration to the fit test concentration. The fit test may then be performed on the test subject.

The concentrations described above are the recommended fit and threshold test concentrations as indicated in OSHA procedures. The fit test devices may include concentrations in a range from above or below these concentrations by 33% in some embodiments. In other embodiments, the fit test devices may comprise concentrations in a range above or below the fit test concentrations and the threshold concentrations by 25% and, in still further embodiments, the fit test devices may comprise concentrations in a range above or below the fit test concentrations and the threshold concentrations by 10%.

Acidic Fit Test Components

An embodiment of the fit test device may comprise a weak solution of non-toxic, irritating acid with a pH of 3 or greater, for example. The weak solution of non-toxic, irritating acid may include, but are not limited to, hydrochloric acid or phosphoric acid in solution. In a specific embodiment, the non-toxic, irritating acid may be stored in a glass or plastic ampule. The ampule may be stored within a reservoir as part of a fit test device. A weak, irritating, non-toxic acid solution with a pH of 3 or greater may be a viable fit test material and glass may be the preferred storage medium.

The embodiments of the described fit test device and method are not limited to the particular embodiments, components, method steps, and materials disclosed herein as such components, process steps, and materials may vary. Moreover, the terminology employed herein is used for the purpose of describing exemplary embodiments only and the terminology is not intended to be limiting since the scope of the various embodiments of the present invention will be limited only by the appended claims and equivalents thereof.

Therefore, while embodiments of the invention are described with reference to exemplary embodiments, those skilled in the art will understand that variations and modifications can be effected within the scope of the invention as defined in the appended claims. Accordingly, the scope of the various embodiments of the present invention should not be limited to the above discussed embodiments, and should only be defined by the following claims and all equivalents.

1. A gas mask fit testing sprayer, comprising:
a reservoir for containing a fit testing composition;
a cap creating an effective air tight seal on the reservoir;
a solvent contained within the reservoir of the gas mask fit test sprayer;
a first sealed ampoule containing a gas mask fit test component, wherein the sealed ampoule is within the reservoir and the gas mask fit test component is soluble in the solvent, wherein a breakable wall of the ampoule separates the solvent from the gas mask fit test component and a resilient wall of the reservoir allows the breakable wall to be broken within the reservoir; and
a sprayer capable of creating a mist of the solvent and the gas mask fit testing component.

2. The gas mask fit testing sprayer of claim 1, wherein the effective air tight seal is created by an adhesive connecting the cap to the reservoir.

3. The gas mask fit testing sprayer of claim 1, wherein the effective air tight seal is created with a gasket or an O-ring between the cap and the reservoir.

4. The gas mask fit testing sprayer of claim 1, wherein the gas mask fit test components are selected from the group consisting of saccharin, denatonium benzoate, a weak solution of non-toxic, irritating acid with a pH of 3 or greater, and isoamyl acetate.

5. The gas mask fit testing sprayer of claim 1, comprising:
a second sealed ampoule, wherein the second ampoule contains a gas generating compound, wherein the gas generating compound reacts with a reactive component of the solvent or a component of the first sealed ampoule.

6. The gas mask fit testing sprayer of claim 5, wherein the gas generating compound is at least one of a carbonate and a bicarbonate and the reactive component is an acid.

7. The gas mask fit testing sprayer of claim 6, wherein the acid is at least one of citric acid, hydrochloric acid, phosphoric acid, and acetic acid.

8. The gas mask fit testing sprayer of claim 5, wherein a reaction between the gas generating compound and the reactive component form a gaseous component within the reservoir thereby increasing, the pressure within the reservoir.

9. The gas mask fit testing sprayer of claim 8, wherein the gaseous component is carbon dioxide.

10. The gas mask fit testing sprayer of claim 1, wherein the reservoir comprises means for breaking the breakable walls of the ampoule.

11. The gas mask fit testing sprayer of claim 1, wherein the reservoir comprises resilient walls that may be squeezed to break the breakable walls of the ampoule.

12. The gas mask fit testing sprayer of claim 1, wherein the first ampoule or the reservoir contains a pressurized gas.

13. The gas mask fit testing sprayer of claim 1, comprising a second ampoule, wherein the second ampoule comprises a pressurized gas.

14. The gas mask fit testing sprayer of claim 1, wherein the first sealed ampoule is made of glass or a plastic.

15. The gas mask fit testing sprayer of claim 1, wherein the sprayer comprises one of a pump sprayer, a pressurized gas sprayer, an aerosol sprayer and an atomizer.

16. A method of fit testing a gas mask, comprising:
breaking a first sealed ampoule containing a gas mask fit test component within a sealed reservoir to release the gas mask fit test component into the reservoir;
spraying the gas mask fit testing component from the sealed reservoir in an area around a person wearing a gas mask;
perceiving an indication from the person wearing the gas mask that the person senses the gas mask fit test component within the gas mask.

17. The method of fit testing a gas mask of claim 16, wherein the reservoir contains a solvent for the gas mask fit testing component.

18. The method of fit testing a gas mask of claim 16, comprising breaking a second sealed ampoule containing a solvent within the sealed reservoir to mix with the gas mask fit test component to produce a fit test composition.

19. The method of fit testing a gas mask of claim 16, wherein spraying the gas mask fit testing component comprises pumping a pump sprayer.

20. The method of fit testing a gas mask of claim 17, wherein one pump of the pump sprayer produces a fine mist comprising the fit test component, wherein the fine mist is between 50 microliters and 300 microliters.

21. The method of fit testing a gas mask of claim 16, comprising breaking a second sealed ampoule within the sealed reservoir to release a gas generating component into the reservoir, wherein the reservoir contains a solvent, the first sealed ampoule and the second sealed ampoule.

22. The method of fit testing a gas mask of claim 21, wherein the first ampoule contains a reactive compound that reacts with the gas generating component to from a gas.

23. The method of fit testing a gas mask of claim 21, wherein the solvent comprises a reactive compound that reacts with the gas generating component to from a gas.

24. A gas mask fit testing device, comprising:
a reservoir for containing a fit testing composition;
a cap creating an effective air tight seal on the reservoir;
a first sealed ampoule containing a gas mask fit test composition, wherein the sealed ampoule is within the reservoir and the breakable walls of the ampoule and the gas mask fit test composition, wherein a breakable wall of the ampoule separates the solvent from the gas mask fit test component from the interior volume of the reservoir; and
an outlet for the gas mask fit testing component to be delivered for a fit test or a threshold test.

25. The gas mask fit testing device of claim 24, wherein the gas mask fit test composition is one of a fit test composition and a threshold test composition.

26. The gas mask fit testing device of claim 24, wherein the fit testing composition comprises one of a solution of isoamyl acetate in water, a solution of denatonium benzoate and sodium chloride in water, a solution of saccharin in water, a non-toxic, irritating weak acid solution with a pH of 3 or greater, anon-toxic, irritating hydrochloric acid solution with a pH of 3 or greater, and a non-toxic, irritating phosphoric acid solution with a pH of 3 or greater.

27. A gas mask fit test device, comprising:
a reservoir for containing a fit testing component;
a cap creating an effective air tight seal on the reservoir;
a solvent contained within the reservoir of the gas mask fit test device;
a first sealed ampoule with a breakable wall, the first sealed ampoule containing a gas mask fit test component, wherein the first sealed ampoule is within the reservoir and the breakable wall of the ampoule separate the gas mask fit test component from the solvent and the gas mask fit test component is soluble in the solvent, wherein a breakable wall of the ampoule separates the solvent from the gas mask fit test component.

28. The gas mask fit test device of claim 27, wherein the solvent is water and the fit test component is isoamyl acetate, having a ratio of between 0.5 part and 2 parts isoamyl acetate per 1,000,000 parts water.

29. The gas mask fit test device of claim 27, wherein the solvent is a sodium chloride solution and the fit test component is denatonium benzoate, having a ratio between 0.5 milligrams to 3.0 milligrams of denatonium benzoate per liter of sodium chloride solution.

30. The gas mask fit test device of claim 27, comprising a second sealed ampoule, wherein the second sealed ampoule containing between 1 gram to 5 grams of denatonium benzoate per liter of sodium chloride solution in the reservoir.

31. The gas mask fit test device of claim 27, wherein the solvent is a sodium chloride solution and the fit test component is denatonium benzoate, having a ratio between 1 gram to 5 grams of denatonium benzoate per liter of sodium chloride solution.

32. The gas mask fit test device of claim 27, wherein the solvent is a water and the fit test component is saccharin, having a ratio of 5 grams to 15 grams of saccharin per liter of water.

33. The gas mask fit test device of claim 27, wherein the solvent is a water and the fit test component is saccharin, having a ratio of 500 grams to the solubility limit of saccharin per liter of water.

34. The gas mask fit test device of claim 27, wherein the solvent is a water and the fit test component is weak acid having a pH of 3 or greater.

35. The gas mask fit test device of claim 27, wherein the reservoir contains a second sealed ampoule and the solvent is contained within the second sealed ampoule.

36. A method of conducting a fit test, comprising:
breaking a first sealed ampoule within a reservoir to release a fit test component to prepare a threshold fit test composition;
spraying the threshold fit test composition from the reservoir in an area around a person to be fit tested for a gas mask;
perceiving an indication from the person to determine whether the person senses the fit test component;
breaking a second sealed ampoule within a reservoir to release more of the fit test component to prepare a fit test composition;
spraying the fit test composition from the reservoir in an area around a person wearing a gas mask; and
determining an indication from the person wearing the gas mask whether the person senses the fit test component within the gas mask.
